(19) United States
(12) Patent Application Publication
(10) Pub. No.: US 2022/0003711 A1
(43) Pub. Date: Jan. 6, 2022

(54) FIELD-EFFECT TRANSISTOR FOR SENSING TARGET MOLECULES

(71) Applicant: Cambridge Enterprise Limited, Cambridge (GB)

(72) Inventors: Ruizhi Wang, Cambridge (GB); Hannah Stern, Cambridge (GB); Stephan Hofmann, Cambridge (GB)

(21) Appl. No.: 17/294,158

(22) PCT Filed: Nov. 15, 2019
(86) PCT No.: PCT/GB2019/053248

§ 371 Date: May 14, 2021

(30) Foreign Application Priority Data
Nov. 16, 2018 (GB) 1818729.4

(51) Int. Cl. G01N 27/414

(52) U.S. Cl. CPC G01N 27/4145; G01N 27/4146

(57) ABSTRACT

A field-effect transistor for sensing target molecules, the field-effect transistor comprising: a substrate; an electric field sensitive layer on the substrate; a hexagonal boron nitride layer comprising a first surface and a second surface, wherein the first surface of the hexagonal boron nitride layer is on the electric field sensitive layer and wherein the second surface of the hexagonal boron nitride layer is functionalized with a plurality of receptor molecules; two or more electrical contacts wherein each of the electrical contacts are in electrical contact with the electric field sensitive layer.

### FIELD AND BACKGROUND

The present techniques relate to the field of sensing target molecules using field-effect transistors. More particularly, they relate to field-effect transistors including a hexagonal boron-nitride layer functionalized with a plurality of receptor molecules.

In recent years there has been an increasing demand for fast and sensitive molecular sensors. In particular, there has been a strong demand for sensors capable of reliably sensing the presence and/or level of allergens, disease causing pathogens, dietary relevant molecules and toxic substances.

A number of techniques can be provided which are capable of sensing the presence of such target molecules including lateral flow tests, enzyme-linked immunosorbent assays (ELISA), gel electrophoresis and blood cultures. However, such techniques typically have low sensitivity (i.e. cannot detect the present of infectious agents until a dangerous level is reached or until an immune response is present) or require a high level of expertise and expense to accurately perform. In addition, many techniques are only capable of detecting the present of a substance and not its level/concentration.

Other techniques can be provided using low-dimensional materials such as graphene or silicon nanowires which can have a high degree of sensitivity while being comparatively easy and inexpensive to use. However, such techniques have proven infeasible to use in practice due to the extreme sensitivity of such materials during manufacture, storage and usage to environmental conditions leading to poor real-world performance and increased costs associated low production yields and short shelf-life.

At least certain embodiments of the present disclosure address one of more of these problems as set out above.

### SUMMARY

Particular aspects and embodiments are set out in the appended claims.

Viewed from one perspective, there can be provided a field-effect transistor for sensing target molecules, the field-effect transistor comprising: a substrate; an electric field sensitive layer on the substrate; a hexagonal boron nitride layer comprising a first surface and a second surface, wherein the first surface of the hexagonal boron nitride layer is on the electric field sensitive layer and wherein the second surface of the hexagonal boron nitride layer is functionalized with a plurality of receptor molecules; two or more electrical contacts wherein each of the electrical contacts are in electrical contact with the electric field sensitive layer.

By including a hexagonal boron nitride layer in this manner, fabrication of the field-effect transistor is simplified as the hexagonal boron nitride layer acts to protect the electric field sensitive layer and hence allows the hexagonal boron nitride layer (i.e. the surface which is to be functionalized) to be aggressively cleaned with a low risk of damage to the electric field sensitive layer. This thereby can allow for both a pristine field electric field sensitive layer to be maintained and for a clean hexagonal boron nitride layer to be prepared which allows for enhanced bonding with the plurality of receptor molecules.

Prior approaches have not attempted to use a protective layer in this manner since the introduction of a conventional dielectric layer between receptor molecules and an electric field sensitive layer can dramatically reduce the electric field strength at the electric field sensitive layer due to the increased distance between the receptor molecules and an electric field sensitive layer and the screening effect of the dielectric. For example, a layer formed of an atomic monolayer of hexagonal boron nitride has a thickness of around 0.34 nm. In contrast, conventional dielectric layers have thicknesses greater than 10 nm.

However, as identified by the present inventors, a material has been recently developed which does not substantially affect electric fields passing through it while still acting as a good insulator. Hexagonal boron nitride is a two-dimensional material which can be made extremely thin, in some examples, down to fewer than ten atomic layers thick while still acting as a good insulator. The hexagon boron nitride layer therefore does not substantially affect an electric field felt at the electric field sensitive layer from the receptor molecules.

Therefore the use of a hexagonal boron nitride layer is able to maintain the sensitivity of the electric field sensitive layer to the receptor molecules while also allowing for both the maintenance of a pristine electric field sensitive layer and enhanced bonding to the plurality of receptor molecules which acts to further enhance the sensitivity of the field-effect transistor to target molecules.

In addition the hexagonal boron nitride layer forms a smooth, well-defined and stable dielectric on the electric field sensitive layer which acts to protect the electric field sensitive layer from environmental degradation during storage and use hence preserving the sensitivity of the field-effect transistor. As a specific example, the hexagonal boron nitride layer acts to passivate the surface of the electric field sensitive layer and protect the electric field sensitive layer from oxidation. Depending on the material used for the electric field sensitive layer, oxides which form on the electric field sensitive layer can be several nanometres thick which would accordingly decrease the sensitivity of the electric field sensitive layer by increasing the distance to the receptor molecules. Further, such oxide layers can be uneven and unstable in some environments (i.e. an oxide might grow or shrink in a particular environment) both of which are detrimental to the reproducibility of measurements made using such devices. Accordingly, the use of a hexagonal boron nitride layer acts to provide both an improvement in the sensitive of the field-effect transistor but also an increase in it stability.

In some examples, each of the plurality of receptor molecules has a binding affinity for the target molecules, and upon interaction between a receptor molecule and a target molecule an electric field is generated thereby gating the electric field sensitive layer. Thereby, the receptor molecules only interact with specific target molecules (i.e. the molecules to which they have a binding affinity) rather than interacting with all, or a large range, of molecules thus ensuring that a signal is only received generated by specific target molecules. Further, by generating an electric field the electric field sensitive layer is directly affected by the interaction between the target molecule and receptor molecule. In some examples the electric field is generated by a change in charge distribution. In other examples the electric field is generated by a change in net charge. It is to be understood that in some examples there may be a pre-existing electric field(s) and the generation of an electric field is an additional electric field which acts on the electric field sensitive layer in addition to the pre-existing electric field(s).

In some examples, the target molecules are charged and upon interaction between the receptor molecule and the target molecule the target molecule becomes bound to the receptor molecule and the change in net charge generates the electric field. Thereby, by changing the net charge (i.e. as opposed to merely changing the charge distribution in the target molecule), an electric field large enough to have a large effect on the electric field sensitive layer is generated. In addition, where the binding is permanent, the field-effect transistor can provide a cumulative measure of how many of the target molecules it has been exposed to. Conversely, where the binding is temporary (e.g. the target molecules spontaneously unbind after a period of time) the field-effect transistor can provide an “instantaneous” measure of the current level/concentration of target molecules and furthermore this allows the reuse of the receptor molecules/field-effect transistor.

In some examples, the plurality of receptor molecules is attached to the hexagonal boron nitride layer using linker molecules. The term “attached” is understood to include any suitable attachment mechanism including ionic bonding, covalent bonding, polar bonding, hydrogen bonding and any other type of non-covalent bonding. Thereby, through the use of linker molecules, a large range of different molecules can be bound to the hexagonal boron nitride layer. In addition, the use of linker molecules can act to prevent interactions between the receptor molecules and the hexagonal boron nitride layer which can thereby enhance the sensitivity of the receptor molecules. In some examples, the linker molecules are: molecules with a polyaromatic hydrocarbon base such as benzene, naphthalene, or pyrene; diaminonaphthalene; pyrenebutanoic acid succinimidyl ester; tetrafulvalene; hexaazatriphenylene-hexacarbonitrile or any other molecule capable of attaching receptor molecules to the hexagonal boron nitride layer.

In some examples, the hexagonal boron nitride layer is modified to allow the plurality of receptor molecules to be directly bonded to the hexagonal boron nitride layer. Thereby, an effect of the electric field from receptor molecules interacting with target molecules on the electric field sensitive layer can be enhanced as the distance between the receptor molecules and the electric field sensitive layer is reduced. In addition, the processing to manufacture the field-effect transistor can be simplified as there is no need to provide a process step of attaching linker molecules to the hexagonal boron nitride layer and a process step of attaching linker molecules to the receptor molecules. As identified by the present inventors, while in principle the electric field sensitive layer could be directly modified to allow the plurality of receptor molecules to be directly bonded to the electric field sensitive layer this would act to damage the electric field sensitive layer and hence reduce its sensitivity to electric fields. Accordingly, by modifying the hexagonal boron nitride layer the receptor molecules can be attached close to the electric field sensitive layer while preserving the pristine characteristics of the electric field sensitive layer.

In some examples, the plurality of receptor molecules comprises one or more types of antibodies and/or one or more types of aptamers and/or one or more types of enzymes and/or one or more types of nucleic acid. Thereby, through the use of antibodies, aptamers, enzymes and nucleic acid, selectivity to a large range of different target molecules can be easily engineered as antibodies, aptamers, enzymes and nucleic acid are available which are selective within a large number of different target molecules. In other words a particular antibody, aptamer, enzyme or nucleic acid may be selective to only a single or small number of target molecule(s) but a large number of different antibodies, aptamers, enzymes and nucleic acid are available. In some examples, through the use of a plurality of types of antibodies and/or aptamers and/or enzymes and/or nucleic acid the overall plurality of receptor molecules can be selective to a specific plurality of different target molecules.

In some examples, the substrate comprises one or more of silicon, silicon dioxide, silicon carbide, aluminium oxide, sapphire, germanium, gallium arsenide (GaAs), an alloy of silicon and germanium, indium phosphide, gallium nitride, polymethyl methacrylate (PMMA), polypropylene carbonate (PPC), polyvinyl butyral (PVB), cellulose acetate butyrate (CAB), polyvinylpyrrolidone (PVP), polycarbonate (PC) and polyvinyl alcohol (PVA). Thereby, readily available materials can be used as the substrate for the device for which established processing techniques may be available. More generally any suitable material can be used for the substrate which can, for example, include conventional semiconductors, polymers and ceramics.

In some examples, the electric field sensitive layer comprises graphene. Thereby, a material with a large change in electrical properties (e.g. resistance) in response to an applied electric field can be provided as the electric field sensitive layer thus providing a high sensitivity to electric fields caused target molecules. In addition, due to the similar lattice spacing and atomic structure of graphene to hexagonal boron nitride, a good adhesion can be obtained between the electric field sensitive layer (graphene) and the hexagonal boron nitride layer while maintaining a large response in the graphene layer and hence the high sensitivity.

In some examples, the electric field sensitive layer comprises one or more of nanowires, nanotubes, and a two-dimensional material. Materials which can form suitable nanowires include, for example, silicon, gallium arsenide (GaAs), indium arsenide (InAs) and Galium Nitride (GaN). Materials which can form suitable nanotubes include, for example, carbon and transition metal dichalcogenide. Materials which can form suitable two dimensional materials include, for example, graphene, phosphorene, silicene, germanene and transition metal dichalcogenides. Thereby, through the provision of low dimensional materials, an electric field sensitive layer which has a high sensitivity to electric fields can be provided.

In some examples, the electric field sensitive layer comprises a bulk semiconductor. Thereby, inexpensive and readily available materials with established processing techniques can be used for the electric field sensitive layer. Examples of suitable semiconductors include: silicon, germanium, gallium arsenide (GaAs), an alloy of silicon and germanium, indium phosphide and gallium nitride. As identified by the present inventors, the use of the hexagonal boron nitride layer can enhance the use of bulk semiconductors as the electric field sensitive layer. Hexagonal boron nitride acts to passivate surfaces from oxidation and hence prevent the formation of native oxides. Many semiconductors, including for example silicon and germanium, form uneven native oxides that are several nanometres thick. These native oxides can be detrimental to both the sensitivity (e.g. by increasing the distance to the receptor molecules) and stability of the electrical field sensitive layer (e.g. by being unstable in certain environments which can lead to the removal of the receptor molecules and hence limit the practical applications). Encapsulation with hexagonal boron nitride allows for a smooth well defined surface dielectric which can be down to atomic thickness. In some examples a single layer of hexagonal boron nitride can be used as the hexagonal boron nitride layer which has a thickness of around 0.34 nanometres thick.

In some examples, the hexagonal boron nitride layer comprises fewer than 10 atomic layers of hexagonal boron nitride. Thereby, by ensuring that the hexagonal boron nitride layer is thin the effect of an electric field caused by target molecules interacting with receptor molecules on the electric field sensitive layer can be strong hence allowing for a good sensitivity of the transistor to target molecules. In some examples, the hexagonal boron nitride layer may comprise 1, 2, 3, 4, 5, 6, 7, 8, 9 or 10 atomic layers of hexagonal boron nitride. In some examples, the hexagonal boron nitride layer may comprise fewer than 2, 5, 10, 20, 50 or 100 atomic layers of hexagonal boron nitride. It will be recognised that in general the fewer the number of atomic layers of hexagonal boron nitride the higher the sensitivity of the device by the increased electric field effect impacting on the electric field sensitive layer. It will also be recognised that the in general the greater the number of atomic layers the greater the electrical insulative effect of the hexagonal boron nitride.

In some examples, the electric field sensitive layer comprises a first surface and a second surface, wherein the first surface of the electric field sensitive layer is on the substrate and wherein the two or more electrical contacts are in electrical contact with the second surface of the electric field sensitive layer. Thereby, through the use of such a “top contact”, a large area can be in electrical contact between the electrical contacts and the electric field sensitive layer hence ensuring a low contact resistance and high sensitivity in electrical measurements made using the electrical contacts and hence to target molecules. In other examples, the electrical contacts are in electrical contact with the same surface of the electrical field sensitive layer that is on the substrate (i.e. the first surface) thereby forming “back contacts”. The use of “back contacts”, can also allow a large area to be in electrical contact between the electrical contacts and the electric field sensitive layer hence ensuring a low contact resistance and high sensitivity in electrical measurements made using the electrical contacts and hence to target molecules. In addition “back contacts” can act to protect the electrical contacts from the environment and to increase the area available for attaching receptor molecules to the hexagonal boron nitride layer.

In some examples, the two or more electrical contacts are in electrical contact with a side portion of the electric field sensitive layer. The use of such “side contacts” in some materials (e.g. graphene and transition metal dichalcogenides) can give rise to a low contact resistance and high sensitivity in electrical measurements made using the electrical contacts and hence to target molecules. In some examples, the electrical contacts will comprise both a “top” and “side” contact which can act to further lower contact resistance and hence increase sensitivity in electrical measurements made using the electrical contacts and hence to target molecules.

In some examples, the two or more electrical contacts comprise one or more of gold, platinum, palladium, copper, titanium, tungsten, nickel, aluminium, molybdenum, chromium, polysilicon, and alloys thereof. Thereby, contacts which have a low electrical resistivity can be provided which can lead to low contact resistance and hence high sensitivity in electrical measurements and further to hence high sensitivity to target molecules. Furthermore, such materials can have established techniques for deposition and device-fabrication hence allowing for inexpensive and accurate manufacturing.

In some examples, the field-effect transistor comprises two electrical contacts in electrical contact with the electric field sensitive layer and wherein the two electrical contacts are arranged to make two-terminal measurements of the electric field sensitive layer. Thereby, a compact arrangement for making electrical measurements can be provided which allows for a high density of field-effect transistors on a chip. Effects of having a plurality of field-effect transistors on a single chip are discussed below. In addition, using two electric contacts can be simple and inexpensive to manufacture.

In some examples, the field-effect transistor comprises four electrical contacts in electrical contact with the electric field sensitive layer and wherein the four electrical contacts are arranged to make four-terminal measurements of the electric field sensitive layer. Thereby, through the use of four-terminal measurements, a high sensitivity in electrical measurements can be obtained as the four-terminal measurement can reduce or eliminate both lead and contact resistance and hence allow for high sensitivity to target molecules.

In some examples, the substrate comprises a first surface and a second surface, wherein the electric field sensitive layer is on the first surface of the substrate, and wherein the field-effect transistor comprises a back-gate on the second surface of the substrate, and wherein the back-gate is arranged to apply a biasing electric field to the electric field sensitive layer. Thereby, the response of the electric field sensitive layer can be tuned. This can allow for the field-effect transistor's sensitivity to target molecules to be enhanced for particular important concentration regimes of the target molecules.

Viewed from one perspective, there can be provided a chip comprising a plurality of any of the field-effect transistors described above. Thereby, a single chip can be provided which allows for a plurality of field-effect transistors to be located in a compact area. In some examples, the plurality of field-effect transistors can be directed either to similar or different purposes to each other.

In some examples, at least two of the plurality of field-effect transistors use the same type of receptor molecule and wherein the chip is arranged to allow for measurements from the at least two of the plurality of field-effect transistors to be multiplexed. Thereby, a high sensitivity to target molecules can be provided, for example, by averaging the signal across a plurality of similar field-effect transistors. Furthermore, multiplexing can lead to a high accuracy in the measurement, for example, as the effect of an atypical field-effect transistor (e.g. a partially defective one) can be reduced.

In some examples, at least two of the plurality of field-effect transistors use different types of receptor molecules which are arranged to interact with different types of target molecule. Thereby, a single compact chip can simultaneously detect the existence or concentrations of a plurality of different target molecules.

Viewed from one perspective, there can be provided a sensing system comprising: any field-effect transistor and/or chip as described above, wherein the field-effect transistor and/or the chip is arranged to be a replaceable element of the sensing system; an electrical measurement module arranged to make electrical measurements on the field-effect transistor and/or the chip; and an output module arranged to output a target molecule measurement.

Thereby, the field-effect transistor and/or chip can be a “consumable” component which allows the system to continue to operate once a given field-effect transistor and/or chip has been exhausted after the “consumed” component has been replaced. The electrical measurement module can be any suitable element capable of making electrical measurements of the field-effect transistor and/or chip. Suitable examples include one or more voltmeters, ammeters and/or ohmmeters. In some examples, the electrical measurement module may include a computing device to process the raw electrical measurements. The output module can be any suitable element capable of outputting a target molecule measurement. In some examples, the output module may include an output device such as a seven-segment display, a monitor, a speaker or a haptic actuator. In some examples, the output module may include a computing device to process raw or processed electrical measurements into a format which may be output on an output device.

Viewed from one perspective, there can be provided a method for sensing target molecules using the system described above, the method comprising: applying a quantity of analyte on the functionalized second surface of the hexagonal boron nitride layer of one of the field-effect transistor(s) of the system; measuring an electrical property of the one of the field-effect transistor(s) of the system using the electrical measurement module; and outputting a target molecule measurement result based on the measured electrical property using the output module.

Thereby, the method may achieve the various effects and advantages described in relation to field-effect transistors above. In some examples, the analyte may be a solid (e.g. a spec of food), liquid (e.g. drinking water) or gaseous (e.g. atmosphere in an enclosed space) thereby allowing measurements to be made in convenient forms of the analyte. In some examples, the target molecule may only be a part of the analyte and the analyte may contain a large number of other substances. In some examples, the analyte is statically applied to the functionalized second surface of the hexagonal boron nitride layer in other examples a continuous or intermittent flow of analyte is passed over the functionalized second surface of the hexagonal boron nitride layer. In some examples, the target molecule measurement result may simply be that the target molecule is present above a threshold concentration. The threshold concentration is dependent on the desired application. For example, a typical threshold concentration when analysing food could be around 0.01 parts per million. In general, the threshold could be, for example, anywhere in range of 1 part per trillion to 100 parts per million. In other examples, the target molecule measurement result may be a numerical concentration level of the target molecule. In general, measured numerical concentrations would be in the range of 1 parts per trillion to 100 parts per million.

In some examples, the electrical property being measured may be voltage, current, resistance, capacitance, impedance or any other suitable electrical parameter.

In some examples, the one of the field-effect transistor(s) of the system comprises two electrical contacts in electrical contact with the electric field sensitive layer and wherein the electrical measurement module measures the electrical property by making a two-terminal measurement of the electric field sensitive layer. Thereby, a compact arrangement for making electrical measurements can be provided which allows for a high density of field-effect transistors on a chip. In addition, using two electric contacts can be simple and inexpensive to manufacture.

In some examples, the electrical property is resistance and wherein the measurement comprises the electrical measurement module determining the resistance by applying one of a current or a voltage across the two electrical contacts and measuring the other of the current or the voltage across the two electrical contacts. Thereby, a straightforward and accurate electrical measurement can be made. In one example device, a current of 10 μA is applied and a voltage of 10 mV is measured. This gives a measured resistance of 1,000Ω. In some examples, the measured resistance may be anywhere in the range between 1Ω and 1,000,000Ω.

In some examples, the one of the field-effect transistor(s) of the system comprises four electrical contacts in electrical contact with the electric field sensitive layer and wherein the electrical measurement module measures the electrical property by making a four-terminal measurement of the electric field sensitive layer. Thereby, through the use of four-terminal measurements, a high sensitivity in electrical measurements can be obtained as the four-terminal measurement can reduce or eliminate both lead and contact resistance and hence allow for high sensitivity to target molecules.

In some examples, the electrical property is resistance and wherein the measurement comprises the electrical measurement module determining the resistance by applying a current across a first pair of the four electrical contacts and measuring a voltage across a second pair of the four electrical contacts. Thereby, the contact and lead resistance can be further reduced thus allowing for more sensitive electrical measurements, and hence more sensitive target molecule measurements, to be made.

In some of either the two or four terminal examples, the current or voltage may be statically applied which allows for a straightforward measurement to be made. In other examples the current or voltage may be applied in an oscillating manner and therefore information on the temporal response of the electrical measurement can be made which. In some examples this oscillating measurement can allow for high sensitivity in the electrical measurement (and hence target molecules) to be made.

In some examples, the system comprises a plurality of field-effect transistors and wherein the electrical measurement module measures the electrical property of each of the plurality of field-effect transistors. Thereby, measurements can be made on a plurality of field-effect transistors which can be directed either to similar or different purposes to each other.

In some examples, at least two of the plurality of field-effect transistors use the same type of receptor molecule and wherein the measured electrical property is multiplexed by the output module between the at least two of the plurality of field-effect transistors which use the same type of receptor molecule. Thereby, a high sensitivity to target molecules can be provided, for example, by averaging the signal across a plurality of similar field-effect transistors. Furthermore, multiplexing can lead to a high accuracy in the measurement, for example, as the effect of an atypical field-effect transistor (e.g. a partially defective one) can be reduced.

In some examples, at least two of the plurality of field-effect transistors use different types of receptor molecules which are arranged to interact with different types of target molecule and wherein the output module outputs at least two target molecule measurement results based on respective measured electrical properties of the at least two of the plurality of field-effect transistors which use different types of receptor molecules. Thereby, a single compact chip can simultaneously detect the existence or concentrations of a plurality of different target molecules.

In some examples, the output module converts the measured electrical property to the target molecule measurement result using a calibration curve. Thereby, a computationally efficient technique for converting from electrical measurements to the target molecule measurement result is provided. In some examples, the calibration curve is generated on a similar device from a “batch” of similar devices. In other examples, the calibration curve is generated on all or part of the device itself. In some examples, the calibration procedure comprises exposing the device being tested to the analyte at one or more known concentrations for one or more time periods.

An example procedure for generating a calibration curve takes a set of devices and subjects each of them to different concentrations of analyte for the same set time. The precise values utilized depend on the specific application and correspond concentrations of interest. An example procedure takes a set of 5 devices which are incubated for 10 min in solutions of analyte concentrations of 0.1 ppb, 1 ppb, 10 ppb, 100 ppb, 1 ppm (one device per solution). The resistance is read out for each device after the pre-defined incubations time (in this example 10 min) and these readings are used to generate a calibration curve of resistance as a function of analyte concentration. It is to be understood that this is merely an example procedure for generating a calibration curve and that any suitable procedure could be used.

Other aspects will also become apparent upon review of the present disclosure, in particular upon review of the Brief Description of the Drawings, Detailed Description and Claims sections.

### BRIEF DESCRIPTION OF THE DRAWINGS

Examples of the disclosure will now be described, by way of example only, with reference to the accompanying drawings in which:

FIG. 1: Schematically illustrates a first example field-effect transistor for sensing target molecules according to teachings of the disclosure.

FIG. 2: Schematically illustrates a second example field-effect transistor for sensing target molecules according to teachings of the disclosure.

FIG. 3A, B: Schematically illustrates example electrical setups which can make A: two-terminal measurements of the electric field sensitive layer and B: four-terminal measurements of the electric field sensitive layer according to teachings of the disclosure.

FIG. 4: Schematically illustrates an example layout of a chip which includes two field-effect transistors according to teachings of the disclosure.

FIG. 5: Schematically illustrates a system according to teachings of the disclosure.

FIG. 6: Schematically illustrates a method for sensing target molecules according to teachings of the disclosure.

FIG. 7: Shows fluorescent microscopy of a hexagonal boron nitride surface with and without fluorescently tagged antibodies.

FIG. 8: Shows a photo of a field-effect transistor according to teachings of the disclosure.

While the disclosure is susceptible to various modifications and alternative forms, specific example approaches are shown by way of example in the drawings and are herein described in detail. It should be understood however that the drawings and detailed description attached hereto are not intended to limit the disclosure to the particular form disclosed but rather the disclosure is to cover all modifications, equivalents and alternatives falling within the spirit and scope of the claimed invention.

It will be recognised that the features of the above-described examples of the disclosure can conveniently and interchangeably be used in any suitable combination.

### DETAILED DESCRIPTION

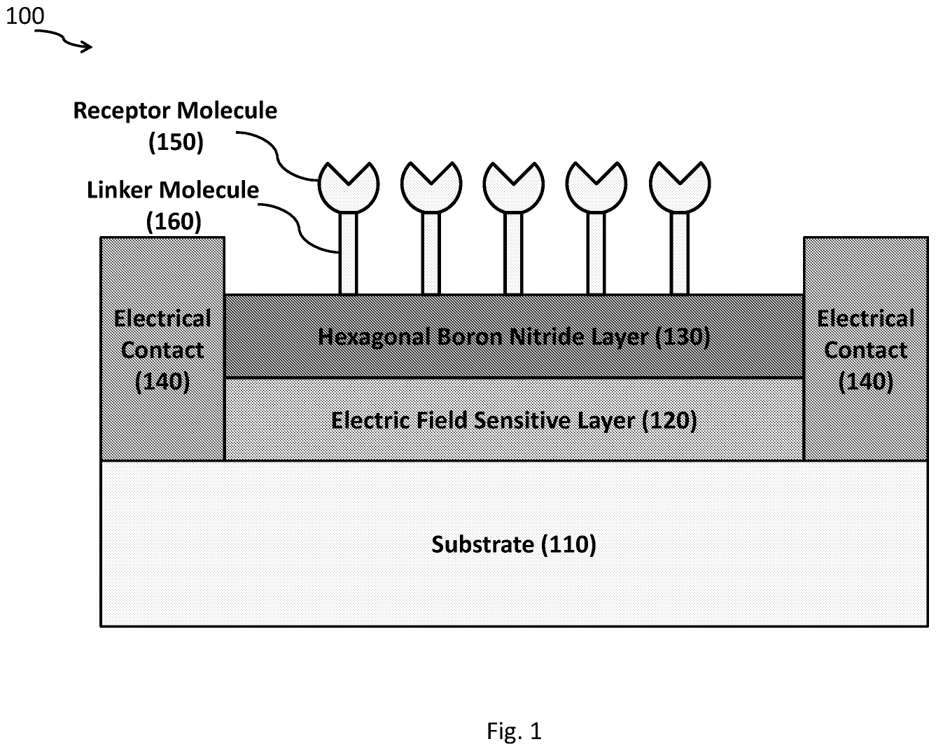

FIG. 1 shows a schematic illustration of a first example field-effect transistor 100 for sensing target molecules according to teachings of the disclosure.

The depicted field-effect transistor 100 includes a substrate 110, an electric field sensitive layer 120, a hexagonal boron nitride layer 130, two electrical contacts 140, a plurality of receptor molecules 150 and a plurality of linker molecule 160.

In the first example depicted in FIG. 1, the substrate 110 is at the bottom of the illustration; with the electric field sensitive layer 120 located on the substrate 110 and the hexagonal boron nitride layer 130 being located on the electric field sensitive layer 120.

The electric field sensitive layer 120 and the hexagonal boron nitride layer 130 are sandwiched in between the two electrical contacts 140. On the upper surface of the hexagonal boron nitride layer 130 are attached a plurality of linker molecules 160 with a plurality of receptor molecules 150 attached on top of the linker molecules 160.

While the field-effect transistor 100 has been depicted in a particular orientation it will be appreciated that the field-effect transistor 100 could operate in any orientation. For example, in use the transistor may be orientated such that the substrate 110 is vertical, or such that the entire transistor is upside-down relative to that depicted.

It will be appreciated that the field-effect transistor 100 may be fabricated using techniques known from semiconductor processing and biopharmaceutical industries.

In some examples, each of the plurality of receptor molecules 150 has a binding affinity for the target molecules. In other words the receptor molecules 150 preferentially bind with target molecules. Upon interaction between a receptor molecule 150 and a target molecule an electric field is generated thereby gating the electric field sensitive layer 120.

In some examples, the target molecules are charged and this change in net charge generates an electric field which affects the proximate electric field sensitive layer 120. In other examples, the target molecules interact with the receptor molecules and/or linker molecules to change a distribution of electric charge hence generating a short-range electric field which affects the proximate electric field sensitive layer 120.

In some examples, upon interaction between the receptor molecule 150 and the target molecule the target molecule becomes permanently bound to the receptor molecule 150. In other examples, upon interaction between the receptor molecule 150 and the target molecule the target molecule becomes temporarily bound to the receptor molecule 150. It will be appreciated, that the time which the target molecule is bound to the receptor molecule 150 can be probabilistic and that the characteristic binding time may be in the range of nanoseconds, microseconds, milliseconds, seconds, minutes, hours, days or longer.

In some examples, the substrate 110 is made from silicon, silicon dioxide, silicon carbide, aluminium oxide, sapphire, germanium, gallium arsenide (GaAs), an alloy of silicon and germanium, indium phosphide, gallium nitride, polymethyl methacrylate (PMMA), polypropylene carbonate (PPC), polyvinyl butyral (PVB), cellulose acetate butyrate (CAB), polyvinylpyrrolidone (PVP), polycarbonate (PC), polyvinyl alcohol (PVA) or any other suitable substrate material. It will be appreciated that, in some examples, the substrate 110 could be made from two or more of the listed materials. For example, part of the substrate 110 could be made from a first material and another part of the substrate 110 could be made from a second material. Additionally or alternatively part or all of the substrate 110 could be made from a mixture of two or more of the materials.

In some examples, the electric field sensitive layer 120 is made from graphene. In other examples, the electric field sensitive layer 120 is made from nanowires, nanotubes, and/or a two-dimensional material. In further examples, the electric field sensitive layer 120 is made from a bulk semiconductor. It will be appreciated that the electric field sensitive layer 120 could be made from a combination of the above-listed materials. For example, the electric field sensitive layer 120 may be made from a first layer of a first material (e.g. an atomic layer of graphene) and a second layer of a second material (e.g. an atomic layer of two-dimensional molybdenum disulfide). Additionally or alternatively, the electric field sensitive layer 120 may be formed from a mixture of two of the above-listed materials. The use of multiple such materials may allow for an improved breadth in sensitivity to impacting electric fields and hence to target molecules.

In some examples, the hexagonal boron nitride layer 130 is made from a few atomic layers of hexagonal boron nitride. The hexagonal boron nitride is a two-dimensional material made from a hexagonal lattice of alternating boron and nitrogen atoms. Hexagonal boron nitride is highly insulating with even a single atomic layer of hexagonal boron nitride acting as a high quality insulator. In addition, hexagonal boron nitride has excellent chemical resistance and mechanical properties including very high strength and hardness. In some examples, the hexagonal boron nitride layer may comprise 1, 2, 3, 4, 5, 6, 7, 8, 9 or 10 atomic layers of hexagonal boron nitride. In some examples, the hexagonal boron nitride layer may comprise fewer than 2, 5, 10, 20, 50 or 100 atomic layers of hexagonal boron nitride.

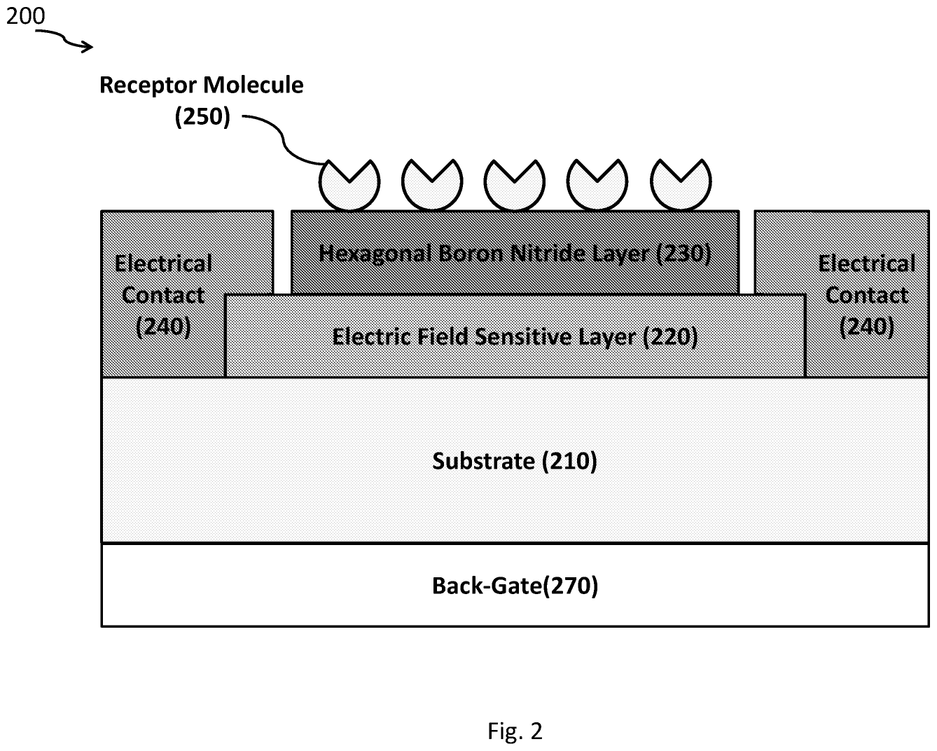

FIG. 2 shows a schematic illustration of a second example field-effect transistor 200 for sensing target molecules according to teachings of the disclosure.

The depicted field-effect transistor 200 includes a substrate 210, an electric field sensitive layer 220, a hexagonal boron nitride layer 230, two electrical contacts 240, a plurality of receptor molecules 250 and a back-gate 270.

It will be appreciated that: the substrate 210 can be substantially similar to the substrate 110 as discussed above; the electric field sensitive layer 220 can be substantially similar to the electric field sensitive layer 120 as discussed above; the hexagonal boron nitride layer 230 can be substantially similar to the hexagonal boron nitride layer 130 as discussed above; and receptor molecule 250 can be substantially similar to receptor molecule 150 as discussed above.

In the second example field-effect transistor 200 depicted in FIG. 2, the back-gate 270 is at the bottom of the illustration, substrate 210 is located on top of the back-gate 270, the electric field sensitive layer 220 is located on top of the substrate 210 and hexagonal boron nitride layer 230 is located on top of the electric field sensitive layer 220.

In the second example field-effect transistor 200 the two electrical contacts 240 contact the electric field sensitive layer 220 both on the top and side surfaces of the electric field sensitive layer 220. On the upper surface of the hexagonal boron nitride layer 230 a plurality of receptor molecules 250 are directly attached.

While the field-effect transistor 200 has been depicted in a particular orientation it will be appreciated that the field-effect transistor 200 could operate in any orientation. For example, in use the transistor may be orientated such that the substrate 210 is vertical, or such that the entire transistor is upside-down relative to that depicted.

It will be appreciated that the field-effect transistor 200 may be fabricated using techniques known from semiconductor processing and biopharmaceutical industries.

In FIG. 1, field-effect transistor 100 is depicted with a plurality of receptor molecules 150 attached to the hexagonal boron nitride layer 130 via a plurality of linker molecules 160. Suitable linker molecules include: polyaromatic hydrocarbon base such as benzene, naphthalene, or pyrene; diaminonaphthalene; pyrenebutanoic acid succinimidyl ester; tetrafulvalene; hexaazatriphenylene-hexacarbonitrile or any other molecule capable of attaching receptor molecules 150 to the hexagonal boron nitride layer 130.

In contrast, the field-effect transistor 200 of FIG. 2 is depicted with a plurality of receptor molecules 250 directly attached to the hexagonal boron nitride layer 230. In order to directly attach the receptor molecules 250 to the hexagonal boron nitride layer 230 in some examples it is necessary to modify the hexagonal boron nitride layer 230 to allow the receptor molecules 250 to bond to the hexagonal boron nitride layer 230. In some examples, the modification may comprise inducing defects in the hexagonal boron nitride layer 230 which allow bonding of the receptor molecules 250. A non-exhaustive list of examples of suitable modification techniques which can allow bonding of receptor molecules 250 include: introduction of mechanical stress/strain to induce cracks in the film; selective etching, for example, via plasma or acid; high-temperature annealing; and electron-beam treatment.

In either case, the receptor molecules may be antibodies and/or aptamers and/or enzymes and/or nucleic acid. These receptor molecules in general will bind to a specific type, or range of, target molecules. In some examples, a range of different antibodies and/or aptamers and/or enzymes and/or nucleic acid may be used as the receptor molecules to allow for selectivity to a desired plurality of different target molecules or ranges of target molecules.

In the example field-effect transistor 100 depicted in FIG. 1, the electric field sensitive layer 120 is electrically side-contacted using the two electrical contacts 140.

In contrast in the example field-effect transistor 200 depicted in FIG. 2, the electric field sensitive layer 220 is contacted both on its top and side surfaces by each of the two electric contacts 240. It will be appreciated that, in some examples, the electric field sensitive layer 120, 220 could be contacted only on its top surface by electrical contacts 140, 240. It will also be appreciated that in some examples, that different of the electrical contacts 140, 240 may contact the electric field sensitive layer 120, 220 on different surfaces to each other (e.g. one electrical contact 140, 240 may contact the electric field sensitive layer 120, 220 on the top and a second electrical contact 140, 240 may contact the electric field sensitive layer 120, 220 on the side) due to the electrical or physical requirements of the particular field effect transistor.

Is some examples, the two or more electrical contacts 140, 240 are made from gold, platinum, palladium, copper, titanium, tungsten, nickel, aluminium, molybdenum, chromium or polysilicon. In some examples, the two or more electrical contacts 140, 240 may be made from an alloy or mixture of two or more of these materials. In some examples, different electrical contacts 140, 240, or portions of the electric contacts 140, 240, may be made of different materials from each other.

In the second example field-effect transistor 200 depicted in FIG. 2, a back-gate 270 is present below the substrate 210. The back-gate is arranged to apply a biasing electric field to the electric field sensitive layer. In some examples, this biasing electric field is generated by applying a voltage to the back-gate 270 relative to the electric field sensitive layer 220.

It is explicitly anticipated that, in some examples, the elements depicted in the first example field-effect transistor 100 and the second example field-effect transistor 200 may be interchanged. As one example, the electrical contacts 240, back-gate 270, and/or receptor molecule(s) 250 without linker molecule(s) may be used with the first example field-effect transistor 100. As another example, the electrical contacts 140 and/or receptor molecule(s) 150 with linker molecule(s) 160 may be used with the second field-effect transistor 200.

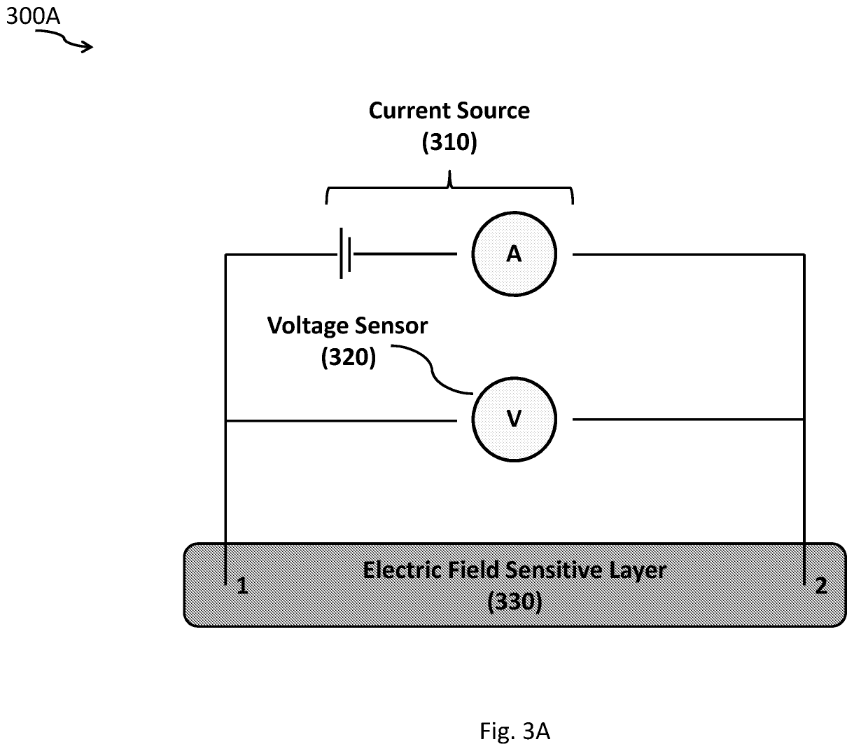

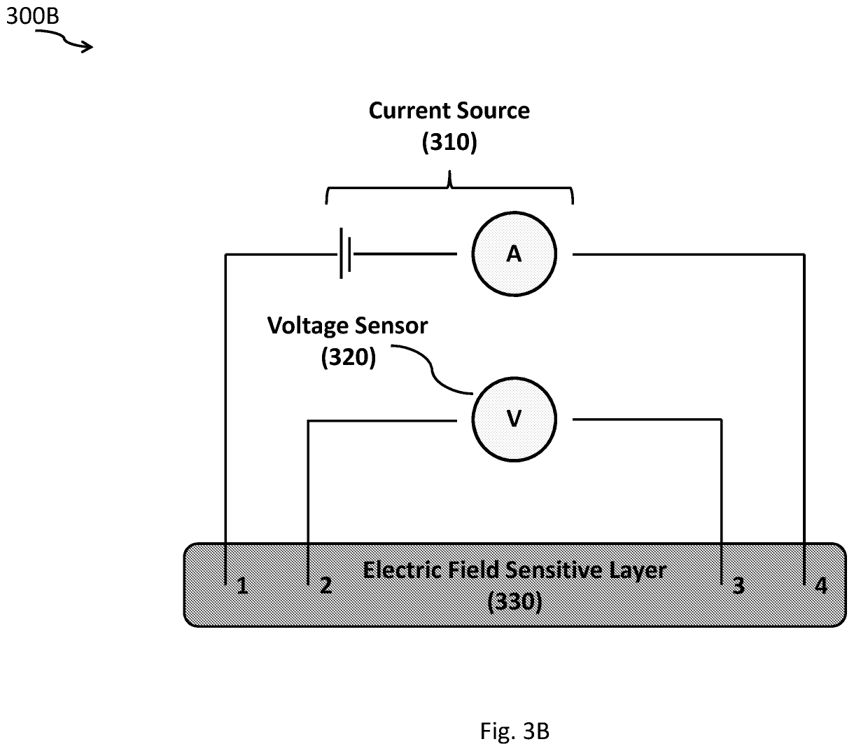

FIGS. 3A and 3B schematically illustrate example electrical setups 300A, 300B for making measurements of an electric field sensitive layer 330 of a field-effect transistor. Both figures depict a current source 310, a voltage sensor 320 and an electric field sensitive layer 330 of a field-effect transistor.

It will be appreciated that the electric field sensitive layer 330 may be substantially similar to electric field sensitive layer 120 within field effect transistor 100 or electric field sensitive layer 220 within field effect transistor 200.

In the depicted examples, current source 310 is made up from a cell or battery and an ammeter. In the depicted examples, voltage sensor 320 is made up from a voltmeter. It will be appreciated that in other examples any suitable electrical measurement equipment could be used to measure a desired electrical parameter of the electric field sensitive layer 330 including using one or more voltmeters, ammeters and/or ohmmeters operating in either a “DC” or “AC” mode.

FIG. 3A schematically illustrates an example “two-terminal” measurement where the same two leads and electrical contacts (1 and 2) on the electric field sensitive layer 330 are used to carry both voltage (i.e. measured from the electric field sensitive layer 330) and current (i.e. supplied from the current source 310). It will be appreciated that in other examples, the voltage may be applied to, and the current measured from, the electric field sensitive layer 330.

In contrast FIG. 3B schematically illustrates an example “four-terminal” measurement which uses separate leads and electrical contacts to carry voltage (i.e. measured from the electric field sensitive layer 330) and current (i.e. supplied from the current source 310). Specifically, the outer set of contacts (1 and 4) are used to supply current to the electric field sensitive layer 330 and the inner set of contacts (2 and 3) are used to measure the voltage from the electric field sensitive layer 330. This arrangement allows for low or negligible lead and contact resistances and accordingly improves the sensitivity of the electrical measurements made.

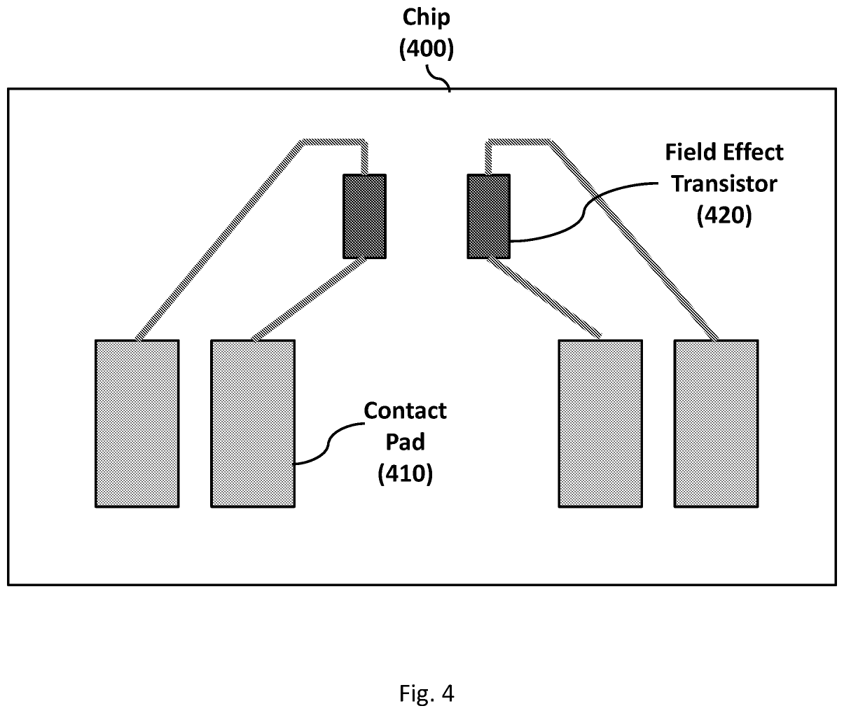

FIG. 4 schematically illustrates an example layout of a chip 400 which includes two field-effect transistors 420. Specifically, the chip 400 has two field effect transistors 420 and four contact pads 410. Two of the contact pads 410 are electrically connected with conductive tracks to each of the two field effect transistors.

The two field-effect transistors 420 can be substantially similar to any of the previously discussed field-effect transistors 100, 200. The contact pads act as comparatively large electrical contact points (i.e. large in comparison to the electric contacts of the field-effect transistors 420) to allow for straightforward connection to electrical measurement equipment for example those depicted in FIGS. 3A and 3B.

In some examples, a chip can have more than two field-effect transistors 420. In some examples, a chip may have 2, 3, 4, 5, 6, 7, 8, 9, 10, 11, 12, 13, 14, 15, 16, 17, 18, 19, 20 or more field-effect transistors 420.

In some examples at least two of the plurality of field-effect transistors 420 use the same type of receptor molecule and wherein the chip is arranged to allow for measurements from the at least two of the plurality of field-effect transistors to be multiplexed. In other words at least two of the plurality of field-effect transistors 420 are sensitive to same target molecules or to the same range of target molecules. The electrical measurements from each of the at least two field-effect transistors 420 can therefore be multiplexed (e.g. averaged or otherwise combined) to obtain a synthesised joint signal and accordingly a synthesised join target molecule measurement.

Additionally or alternatively, in some examples, at least two of the plurality of field-effect transistors use different types of receptor molecules which are arranged to interact with different types of target molecule. In other words the at least two of the plurality of field-effect transistors 420 are sensitive to different target molecules or different ranges of target molecules. The electrical measurements from each of the at least two field-effect transistors 420 can therefore, in some examples, be used to obtain separate target molecule measurements for a plurality of different types of target molecules. In some examples, the different types of receptor molecules may be sensitive to the same target molecules but respond differently to different concentrations of target molecules thereby providing further information on the concentration of the target molecule.

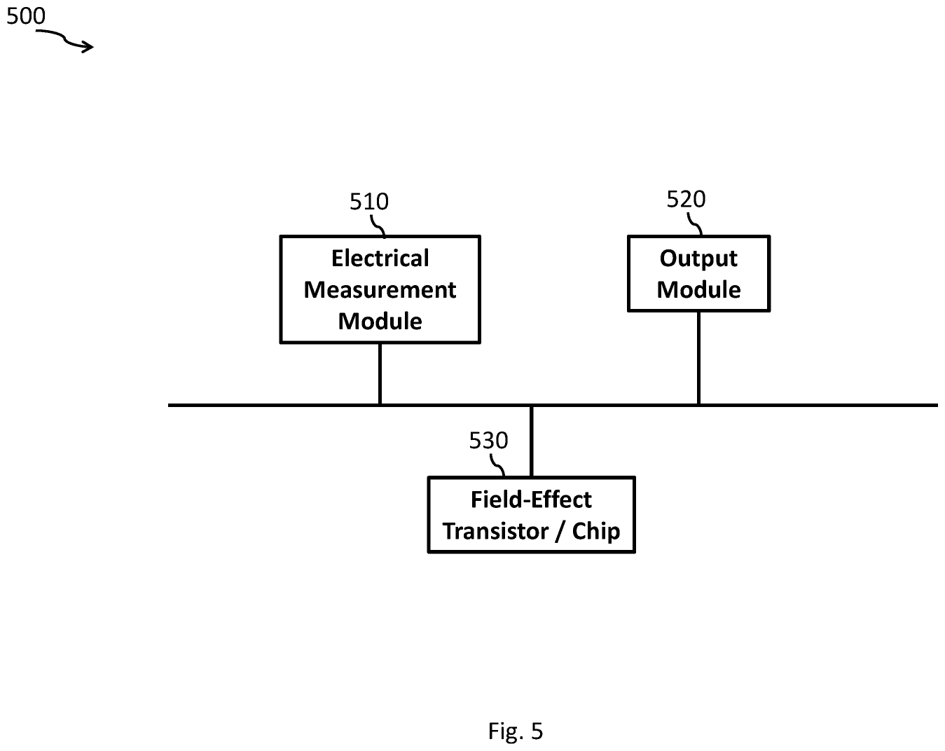

FIG. 5 schematically illustrates a system 500 which includes an electrical measurement module 510 an output module 520 and a field-effect transistor/chip 530. The field-effect transistor/chip 530 may include one or more field-effect transistors which are substantially similar to field-effect transistors 100, 200 or 420 discussed above and may additionally or alternatively include a chip which is substantially similar to chip 400 as discussed above.

In some examples, the field-effect transistor/chip 530 is designed to be an easily replaceable component of the system 500. This can be particularly useful if the field-effect transistor/chip 530 has a short effective lifetime than the overall system 500.

The electrical measurement module 510 is arranged to make electrical measurements on the field-effect transistor/chip 530. In some examples, the electrical measurement module may include a computing device to process raw electrical measurements. In some examples, the electrical measurement module includes the measurement arrangement shown in FIG. 3A or 3B.

The output module 520 is arranged to output a target molecule measurement. In some examples, the output module may include an output device such as a seven-segment display, a monitor, a speaker or a haptic actuator. In some examples, the output module may include a computing device to process raw or processed electrical measurements into a format which may be output on an output device.

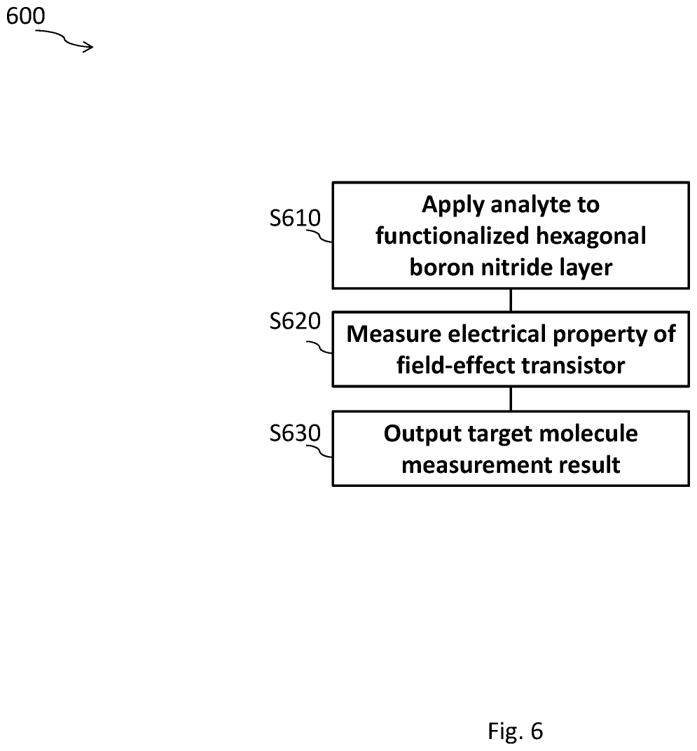

FIG. 6 schematically illustrates a method 600 for sensing target molecules using a system. The system can be substantially similar to the system 500 described above.

At step S610, a quantity (e.g. a drop) of analyte is applied on the functionalized second surface of a hexagonal boron nitride layer 130, 230 of one of the field-effect transistor(s) 100, 200 of the system 500.

At step S620, an electrical property of the one of the field-effect transistor(s) 100, 200 of the system 500 is measured using the electrical measurement module 510.

In some examples, the one of the field-effect transistor(s) 100, 200 of the system comprises two electrical contacts in electrical contact with the electric field sensitive layer 120, 220 and wherein the electrical measurement module 510 measures the electrical property by making a two-terminal measurement of the electric field sensitive layer 120, 220. In some examples, the electrical property is resistance and wherein the measurement comprises the electrical measurement module 510 determining the resistance by applying one of a current or voltage across the two electrical contacts and measuring the other of the current or the voltage across the two electrical contacts.

In other examples, the one of the field-effect transistor(s) 100, 200 of the system 500 comprises four electrical contacts in electrical contact with the electric field sensitive layer and wherein the electrical measurement module 510 measures the electrical property by making a four-terminal measurement of the electric field sensitive layer. In some examples, the electrical property is resistance and wherein the measurement comprises the electrical measurement module 510 determining the resistance by applying a current across a first pair of the four electrical contacts and measuring a voltage across a second pair of the four electrical contacts.

In some examples, the system 500 comprises a plurality of field-effect transistors 100, 200 and wherein the electrical measure module 510 measures the electrical property of each of the plurality of field-effect transistors.

At step S630, a target molecule measurement result is output based on the measured electrical property using the output module 520.

In some examples, where the system 500 comprises a plurality of field-effect transistors 100, 200, at least two of the plurality of field-effect transistors 100, 200 use the same type of receptor molecule and wherein the measured electrical property is multiplexed by the output module 520 between the at least two of the plurality of field-effect transistors which use the same type of receptor molecule.

Additionally or alternatively, in some examples, at least two of the plurality of field-effect transistors 100, 200 use different types of receptor molecules which are arranged to interact with different types of target molecule and wherein the output module outputs 520 at least two target molecule measurement results based on respective measured electrical properties of the at least two of the plurality of field-effect transistors 100, 200 which use different types of receptor molecules.

In some examples, the output module 520 converts the measured electrical property to the target molecule measurement result using a calibration curve.

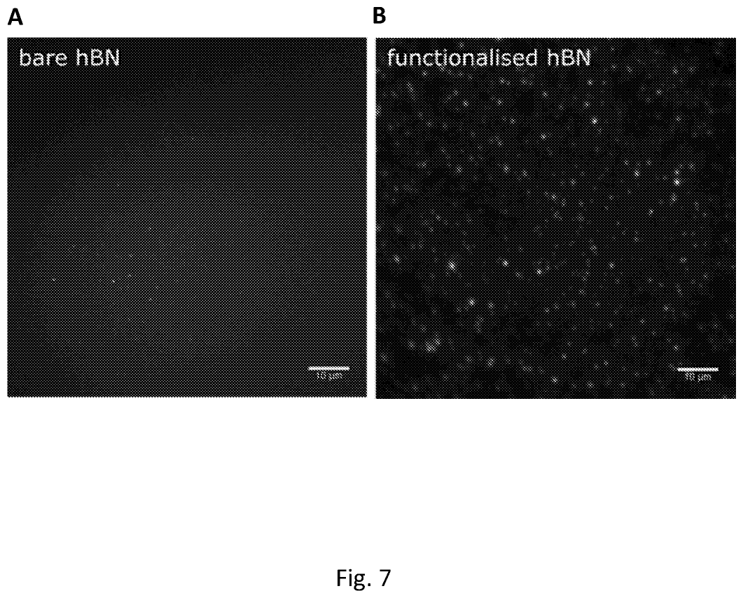

FIG. 7 shows a pair of images taken using fluorescent microscopy of a hexagonal boron nitride surface before and after addition of antibodies. The antibodies are fluorescently tagged such that they can be seen under fluorescent microscopy. As can be seen far more bright points are visible (i.e. fluorescing antibodies) in FIG. 7B than in FIG. 7A. This demonstrates that a large number of antibodies have been successfully immobilized on the hexagonal boron nitride surface and accordingly that the hexagonal boron nitride surface has been successfully functionalized.

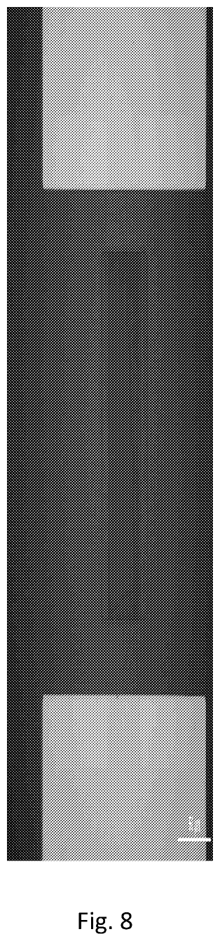

FIG. 8: Shows a photo of a field-effect transistor similar to field-effect transistors 100, 200, 420 described above. The photo is a top “plan” view of the field-effect transistor. The background portion shows the substrate with electrical contacts clearly visible at the top and bottom of the photo. A thin strip of hexagonal boron nitride on top of graphene (acting as an electric field sensitive layer) is just visible bridging the two electrical contacts. A central region to which “functionalizing” receptor molecules have been applied is visible in a central rectangle overlapping the thin strip.

The methods discussed above may be performed under control of a computer program executing on a device. Hence a computer program may comprise instructions for controlling a device to perform any of the methods discussed above. The program can be stored on a storage medium. The storage medium may be a non-transitory recording medium or a transitory signal medium.

In the present application, the words “arranged to . . . ” are used to mean that an element of an apparatus has a configuration able to carry out the defined operation. In this context, an “arrangement” means a configuration or manner of interconnection of hardware or software. For example, the apparatus may have dedicated hardware which provides the defined operation, or a processor or other processing device may be programmed to perform the function. “Arranged to” does not imply that the apparatus element needs to be changed in any way in order to provide the defined operation.

Although illustrative teachings of the disclosure have been described in detail herein with reference to the accompanying drawings, it is to be understood that the invention is not limited to those precise teachings, and that various changes and modifications can be effected therein by one skilled in the art without departing from the scope and spirit of the invention as defined by the appended claims.

1. A field-effect transistor for sensing target molecules, the field-effect transistor comprising:
a substrate;
an electric field sensitive layer on the substrate;
a hexagonal boron nitride layer comprising a first surface and a second surface,
wherein the first surface of the hexagonal boron nitride layer is on the electric field sensitive layer and wherein the second surface of the hexagonal boron nitride layer is functionalized with a plurality of receptor molecules;
two or more electrical contacts wherein each of the electrical contacts are in electrical contact with the electric field sensitive layer.

2. The field-effect transistor of claim 1, wherein each of the plurality of receptor molecules has a binding affinity for the target molecules, and wherein upon interaction between a receptor molecule and a target molecule an electric field is generated thereby gating the electric field sensitive layer.

3. The field-effect transistor of claim 2, wherein the target molecules are charged and wherein upon interaction between the receptor molecule and the target molecule the target molecule becomes bound to the receptor molecule and the change in net charge generates the electric field.

4. The field-effect transistor of claim 1, wherein the plurality of receptor molecules is attached to the hexagonal boron nitride layer using linker molecules.

5. The field-effect transistor of claim 1, wherein the hexagonal boron nitride layer is modified to allow the plurality of receptor molecules to be directly bonded to the hexagonal boron nitride layer.

6. The field-effect transistor of claim 1, wherein the plurality of receptor molecules comprises one or more types antibodies and/or one or more types of aptamers and/or one or more types of enzymes and/or one or more types of nucleic acid.

7. (canceled)

8. The field-effect transistor of claim 1, wherein the electric field sensitive layer comprises one or more of graphene, nanowires, nanotubes, a two-dimensional material, or a bulk semiconductor.

9. (canceled)

10. (canceled)

11. The field-effect transistor of claim 1, wherein the hexagonal boron nitride layer comprises fewer than 10 atomic layers of hexagonal boron nitride.

12. The field-effect transistor of claim 1, wherein the electric field sensitive layer comprises a first surface and a second surface, wherein the first surface of the electric field sensitive layer is on the substrate and wherein the two or more electrical contacts are in electrical contact with the second surface of the electric field sensitive layer.

13. (canceled)

14. (canceled)

15. The field-effect transistor of claim 1, wherein the field-effect transistor comprises two electrical contacts in electrical contact with the electric field sensitive layer and wherein the two electrical contacts are arranged to make two-terminal measurements of the electric field sensitive layer, or wherein the field-effect transistor comprises four electrical contacts in electrical contact with the electric field sensitive layer and wherein the four electrical contacts are arranged to make four-terminal measurements of the electric field sensitive layer.

16. (canceled)

17. The field-effect transistor of claim 1, wherein the substrate comprises a first surface and a second surface, wherein the electric field sensitive layer is on the first surface of the substrate, and
wherein the field-effect transistor comprises a back-gate on the second surface of the substrate, and wherein the back-gate is arranged to apply a biasing electric field to the electric field sensitive layer.

18. A chip comprising a plurality of field-effect transistors according to claim 1, wherein at least two of the plurality of field-effect transistors use the same type of receptor molecule and wherein the chip is arranged to allow for measurements from the at least two of the plurality of field-effect transistors to be multiplexed, or wherein at least two of the plurality of field-effect transistors use different types of receptor molecules which are arranged to interact with different types of target molecule.

19. (canceled)

20. (canceled)

21. A sensing system comprising:
a field-effect transistor according to claim 1, wherein the field-effect transistor is arranged to be a replaceable element of the sensing system;
an electrical measurement module arranged to make electrical measurements on the field-effect transistor; and
an output module arranged to output a target molecule measurement.

22. A method for sensing target molecules using the sensing system of claim 21, the method comprising:
applying a quantity of analyte on the functionalized second surface of the hexagonal boron nitride layer of the field-effect transistor of the system;
measuring an electrical property of the field-effect transistor of the system using the electrical measurement module; and
outputting a target molecule measurement result based on the measured electrical property using the output module.

23. (canceled)

24. (canceled)

25. The method of claim 22, wherein the field-effect transistor of the system comprises four electrical contacts in electrical contact with the electric field sensitive layer and wherein the electrical measurement module measures the electrical property by making a four-terminal measurement of the electric field sensitive layer, wherein the electrical property is resistance and wherein the measurement comprises the electrical measurement module determining the resistance by applying a current across a first pair of the four electrical contacts and measuring a voltage across a second pair of the four electrical contacts.

26. (canceled)

27. The method of claim 22, wherein the system comprises a plurality of field-effect transistors and wherein the electrical measure module measures the electrical property of each of the plurality of field-effect transistors.

28. The method of claim 27, wherein at least two of the plurality of field-effect transistors use the same type of receptor molecule and wherein the measured electrical property is multiplexed by the output module between the at least two of the plurality of field-effect transistors which use the same type of receptor molecule.

29. The method of claim 27, wherein at least two of the plurality of field-effect transistors use different types of receptor molecules which are arranged to interact with different types of target molecule and wherein the output module outputs at least two target molecule measurement results based on respective measured electrical properties of the at least two of the plurality of field-effect transistors which use different types of receptor molecules.

30. The method of claim 22, wherein the output module converts the measured electrical property to the target molecule measurement result using a calibration curve.

31. A method for sensing target molecules, the method comprising:
applying a quantity of analyte on a functionalized second surface of a hexagonal boron nitride layer of a field-effect transistor for sensing target molecules, the field-effect transistor comprising an electric field sensitive layer on a substrate and the hexagonal boron nitride layer, the hexagonal boron nitride layer comprising a first surface and the second surface, the first surface of the hexagonal boron nitride layer being on the electric field sensitive layer and the second surface of the hexagonal boron nitride layer being functionalized with a plurality of receptor molecules;
measuring an electrical property of the field-effect transistor using two or more electrical contacts wherein each of the electrical contacts are in electrical contact with the electric field sensitive layer; and
outputting a target molecule measurement result based on the measured electrical property.
